(19) United States
(12) Patent Application Publication
(10) Pub. No.: US 2009/0181123 A1
(43) Pub. Date: Jul. 16, 2009

(54) METHODS FOR ENHANCING WINE FLAVOR

(76) Inventor: Daniel La Caze, Rolling Hills, CA (US)

Correspondence Address: GRANT ANDERSON LLP;C/O PORTFOLIOIP, P.O. BOX 52050, MINNEAPOLIS, MN, 55402, US

(21) Appl. No.: 12/138,697

(22) Filed: Jun. 13, 2008

Related U.S. Application Data
(60) Provisional application No. 60/943,669, filed on Jun. 13, 2007.

(51) Int. Cl. C12G 1/00; C12N 1/19

(52) U.S. Cl. 426/15; 426/61; 435/254.2

(57) ABSTRACT

The present invention relates generally to methods and compositions for enhancing the flavor or other properties of wine, and wine products produced by such methods.

### PRIORITY

Priority is claimed to U.S. Provisional Application Ser. No. 60/943,669, filed Jun. 13, 2007, and entitled Methods for Enhancing Wine Flavor, which is referred to and incorporated herein by reference in its entirety.

### FIELD OF THE INVENTION

The invention pertains generally to novel methods and compositions for enhancing the flavor or other properties of wine, and wine products produced by such methods.

### BACKGROUND

Making wine involves a series of production steps that can be broadly broken down into two major sets: (1) cultivation, which includes growing and harvesting suitable grapes, and (2) winemaking, which involves processing the grapes into a liquid mixture, fermenting such mixture with yeast, and later-stage processing, including, but not limited to, some or all of the following steps: yeast inoculation, acid and SO₂ adjustment, bacterial inoculation, oak barrel aging, fining, and mixing. A number of process variations can be implemented during these steps to alter the final taste of the wine. The flavor characteristics that distinguish different wines, both within a class of wines (clarets, for example) and between classes of wines, are due to the presence and relative concentration of certain natural products from the grapes or from the fermentation process.

Wine comprises primarily water and alcohol, but the following solutes are present in the following approximate concentrations:

TABLE 1

| Component | Average Concentration |
|---|---|
| Glycerol | 7000 |
| Acid | 6000 |
| Broad-range Phenols | 1800 |
| Minerals | 1200 |
| Sugars | 750 |
| Amino Acids | 550 |
| Higher Alcohols | 500 |
| Volatile Acidity | 400 |
| Sorbitol & Mannitol | 300 |
| Sulfites | 80 |
| Acetaldehyde | 70 |
| Esters | 60 |

It is primarily the combination and relative concentrations of these major components that impart the flavor characteristics for various wines. Furthermore, there is great diversity within the certain classes of components, especially within the broad-range phenols and esters groups. The broad-range phenols class encompasses sub-groups, such as terpenes, monoterpenes, isoprenoids (some further sub-groups include norisoprenoids, sequeterpines, isoprenoids) and aromatics, as well as alcohols and esters and concatemers (such as tannins) thereof. It is the grapes, the fermentation process, and the winemaking processes (such as oaking and ageing) that contributes these components and adjusts their relative concentrations in wine.

Though the presence and concentrations of some of components above can be reasonably controlled through certain fermenting and winemaking processes, most components contributed by the grapes (natural products such as broad-range phenols, esters and certain acids) cannot be further adjusted nor added by the winemaker during the winemaking process. The presence of these natural products and their relative concentrations is entirely dictated by the composition of the grapes, which in turn, is a product of the grape varietal (genetic makeup) and the conditions present during the growing season that affect the grape varietal's gene expression. Such natural products are produced by certain enzymatic reactions within the plant.

These natural products profoundly affect a wine's taste, and are typically the distinguishing factor between different wine types (Claret and Pinot Grigio, for example) and relative concentrations of such products between wines within a certain class affect a wine's rating (Wine Spectator). The former distinction is primarily due to the presence or lack of certain natural products and the later is primarily due to the relative concentrations of similar groups of natural products (although the presence of unique groups may also play a smaller role). Due to these conditions, the wine industry has worked for many years to identify certain varietals and growing locations that together will provide best combination of natural product production (on average) for a certain types of wine. Over the years, certain locations, such as Bordeaux, Napa, and Montecello to name a few, have become prime grape growing locations for certain varietals, and hence real estate prices in such areas have increased dramatically.

Current methods employed by winemakers to improve the flavor characteristics of wine, include altering the grape cultivation and/or winemaking processes and choosing to mix/blend wines from different varietals. Although crude and somewhat inefficient, such techniques allow winemakers to produce wine with certain desired characteristics. Unfortunately, not all characteristics are easy to replicate year to year and sometimes require significant investment to produce.

Various flavor-enhancing plant natural products have been identified and their biosynthetic pathways characterized. Plant volatile biosynthetic enzymes have been identified, including those involved in terpene synthesis. {Pichersky, E., et al., Science 311:808-811 (2006)). Phenyl propenes that have anti-microbial properties and help to attract pollinators are produced by plants. Phenyl propenes include, for example, eugenol and isoeugenol. Plant materials containing these compounds have been used by humans to preserve and flavor their food. These plant materials include, for example, basil, cloves, and allspice. Enzymes that are involved in the biosynthetic pathways to produce phenylpropenes, and their corresponding DNA sequences, have been identified. (Koeduka, T., et al., Proc. Nat'l. Acad. Sci. USA 103:10128-10133 (2006)). Other flavor-enhancing plant natural products include, for example, those present in wood, such as oak, or other woods used in wine processing, that add flavor to wine.

A recombinant wine yeast strain expressing the gene coding for beta-(1,4)-endoxylanase from Asperigillus nidulans under the control of the yeast actin promoter has been found to secrete active xylanase enzyme into the culture medium. (Ganga, M. A., et al., Int. J. Food Microbiol. Vol. 47:171-8 (1999)) A recombinant yeast strain carrying a recombinant genetic insert including a DUR1,2 gene was developed by inserting the genetic insert into a strain commonly used in the wine industry, for the purpose of increasing the expression of urea amidolyase. (Agency Response Letter GRAS Notice No. GRN 000175, Jan. 6, 2006, CFSAN/Office of Food Additive Safety). A recombinant yeast strain expressing the gene encoding L(+)-lactate dehydrogenase from Lactobacillus casei has been developed; yeast expressing the gene convert glucose to both ethanol and lactate. (Dequin, S. and Barre, P., Biotechnology (NY) 2:173-7 (1994)).

Processes and technologies that will allow wine producers to further control and consistently configure the taste of their wine while reducing the associated production costs, including real estate investment, are greatly sought after. Such technologies would allow winemakers to produce high quality wines, for example, wines having high Wine Spectator ratings, such as, for example, higher than 80, 85, 90, or 95, at a fraction of the cost of the current high-end brand wines.

### DESCRIPTION

The details of one or more embodiments of the invention are set forth in the description below. Other features, objects, and advantages of the invention will be apparent from the description, and from the claims.

Provided in the present invention are methods for enhancing the flavor of wine comprising the use of various recombinant yeast strains that are engineered to produce the various natural products that grapes contribute to the wine, thereby either introducing such compounds to the wine or increasing their relative concentration within wine if already present. These natural products include, but are not limited to, the natural products listed in Table 2, such as, for example, esters, isoprenoids, phenols, acids, pyrazines, and combinations and variations thereof (alcohol esters and phenol concatemers, for instance) that are produced via various natural enzymatic pathways. The transformed yeast used to create these natural products shall be engineered to express such genes responsible for a particular type of natural product or products, usually with a tendency toward over-expression and production of such natural product.

In certain embodiments of the invention, the recombinant yeast strains are used to ferment grape juice, secreting natural products into the grape juice, and producing a wine that is concentrated in one or more natural products, compared to wine that is produced without the recombinant yeast. The concentrated wine may either be used alone for consumption, or the concentrated wine may then be added to wine that does not have enhanced levels of the secreted natural product, creating through this mixing a wine that has an increased, and desirable, amount of the natural product. More than one concentrated wine may be produced, and combined in different mixtures, with non-concentrated wine. Or, a concentrated wine may be mixed with another concentrated wine. Each of the concentrated wines may, for example, have different properties due to the concentration of different natural products. By mixing smaller batches of the natural product concentrated wine with the non-concentrated wine, or smaller batches of various concentrated wines, appropriate ratios of the wines can be determined to provide guidance for the scale-up blending and production of larger batches of enhanced flavor wine.

In other embodiments of the present invention, the addition of natural products to wine, using the methods of the present invention, may enhance properties other than the flavor of the wine. For example, anti-oxidants, or other health components may be added to enhance the healthful properties of wine.

In some embodiments of the invention, the recombinant yeast strain used to produce concentrated wine expresses two or more heterologous genes. In some examples, the two or more heterologous genes code for enzymes from the same metabolic pathway to produce a natural product. Or, for example, the two or more heterologous genes may cause the production of more than one natural product.

In other embodiments of the invention, two or more recombinant yeast strains may be used to produce concentrated wine, each expressing a different heterologous gene. In some embodiments, two or more recombinant yeast strains may be used to produce concentrated wine, where at least one expresses more than one heterologous gene. The two or more recombinant yeast strains may produce enzymes from the same metabolic pathway to produce a natural product. Or, for example, the two or more recombinant yeast strains may cause the production of more than one natural product. Also included within the scope of the invention is the use of one or more recombinant yeast strains along with a non-recombinant yeast strain to produce a concentrated wine of the invention.

In other embodiments of the present invention are wines produced using the methods of the present invention. These wines may include, for example, the concentrated wines, the mixed concentrated wines, and the concentrated wine mixed with non-concentrated wines of the present invention.

Those of ordinary skill in the art would recognize that the methods described herein may also be applied to modifying the characteristics of other fermented products, such as, for example, beer. The methods of the present invention may be used to add natural products to beer during fermentation. Also, those of ordinary skill in the art recognize that organisms other than yeast, such as, for example, bacteria, may be used during the fermentation process to produce natural products. For example, bacteria have been used for malo-lactic acid fermentation during wine fermentation (Pilone, G. J., et al., Appl. Microbiol. 14:608-15 (1966); Jussier, D., et al., Appl. Environ. Microbiol. 72: 221-27 (2006)). Genes used for synthesizing natural products may be expressed in such bacteria, and included during fermentation.

Thus, the present invention provides a method of producing wine, comprising mixing at least two fermented grape juices, wherein at least one of the fermented grape juices is fermented by recombinant yeast transformed with a DNA sequence comprising a heterologous gene, wherein the expression of the heterologous gene causes the yeast to secrete a flavor-enhancing natural product.

In other embodiments of the present invention, a method is provided for enhancing the flavor of wine, comprising fermenting grape juice using a recombinant yeast strain that secretes a natural product to obtain an enhanced wine concentrate.

In other embodiments, a method is provided for enhancing the flavor of wine, comprising fermenting grape juice using a recombinant yeast strain that secretes a natural product to obtain an enhanced wine concentrate; and adding the enhanced wine concentrate to wine to produce an enhanced-flavor wine. In some aspects, more than one enhanced wine concentrate is added to the wine, wherein the recombinant yeast strains used to produce each of the enhanced wine concentrates are different.

In certain embodiments, the methods for producing the wine of the present invention also comprise titrating different amounts of at least one of the enhanced wine concentrates into individual portions of wine; and testing each portion of wine to determine the ratio of enhanced wine concentrates and wine that has an enhanced flavor. Testing may, for example, comprise tasting. In some aspects, larger batches of enhanced flavor wine may be prepared using the ratios determined from this method.

The present invention also provides a method of increasing the concentration of a natural product in wine, comprising fermenting grape juice with a recombinant yeast transformed with a DNA sequence comprising a heterologous gene, wherein the expression of the heterologous gene causes the yeast to secrete the natural product; and adding the fermented grape juice to wine, thereby increasing the concentration of the natural product in the wine.

Also provided in the present invention is a method of adding an oak barrel flavor to a wine, comprising fermenting grape juice with a recombinant yeast transformed with a DNA sequence comprising a heterologous gene, wherein the expression of the heterologous gene causes the yeast to secrete a natural product present in oak. The wine may be, for example, fermented in an oak barrel. Or, the wine may, for example, not be fermented in an oak barrel.

In other embodiments of the present invention, a method is provided for producing wine, comprising fermenting grape juice with a recombinant yeast transformed with a DNA sequence comprising a heterologous gene, wherein the expression of the heterologous gene causes the yeast to secrete a natural product; and aging the fermented grape juice.

Also provided in the present invention is wine produced by any of the methods of the present invention. The present invention also provides a wine comprising a natural product secreted by recombinant yeast, wherein the recombinant yeast are transformed with a DNA sequence comprising a heterologous gene, wherein the expression of the heterologous gene causes the recombinant yeast to secrete the natural product; and the natural product was introduced to the wine during fermentation in the presence of the recombinant yeast. The present invention also provides a wine comprising a natural product secreted by recombinant microbes other than yeast, such as, for example, bacteria, wherein the recombinant microbes are transformed with a DNA sequence comprising a heterologous gene, wherein the expression of the heterologous gene causes the recombinant microbes to secrete the natural product; and the natural product was introduced to the wine during fermentation in the presence of the recombinant microbes.

In other embodiments of the present invention, recombinant yeast is provided, comprising a DNA sequence comprising a heterologous gene, wherein the expression of the gene causes the yeast to secrete a natural product. In some aspects, the natural product is secreted during fermentation.

In the various embodiments of the present invention, the heterologous gene may, for example, encode a natural product, or the gene may encode a protein that produces a natural product. In some aspects of the invention, the yeast is transformed with a recombinant plasmid vector, comprising the DNA sequence. In other aspects, the recombinant yeast is transformed with a DNA sequence comprising at least two heterologous genes, wherein the expression of the heterologous genes causes the yeast to secrete at least two different natural products. The recombinant yeast may, for example, be transformed with at least two recombinant plasmid vectors, each of which comprises a different heterologous gene encoding a different natural product.

In the embodiments of the present invention, the natural product secreted by the yeast may be, for example, a product that is produced by a grape plant. In certain aspects, the natural product is a product that is present in grapes. In other aspects of the present invention, the natural product is a product that is present in wood, for example, but not limited to, oak wood. In certain embodiments of the invention, the natural product is selected from the group consisting of lactones, eugenol, vanillin, vanillyl alcohol, cyclotene, ethyoxylactone, maltol, hydroxymaltol, dihydromaltol, syringaldehyde, sinapaldehyde, furfural, lignin, hemi-cellulose, and tannin. The lactone may be, for example, but not limited to, a methyl-octalactone, for example, alpha-methyl-beta-octalactone. The tannin may be, for example, but not limited to, a gallitannin or an ellagitannin. In other aspects of the invention, the natural product is selected from the group consisting of acids, terpenes, norisoprenoids, esters, isoprenoids, phenols, acids, and pyrazines. In other aspects, the natural product is selected from the list of natural products of Table 2.

In some embodiments of the present invention, at least 2 fermented grape juices are mixed, and two of the grape juices are fermented by recombinant yeast transformed with a DNA sequence comprising a heterologous gene, wherein the expression of the gene cause each recombinant yeast to secrete a different flavor-enhancing natural product. The grape juice may be, for example, fermented by recombinant yeast in a separate vessel.

These and other embodiments are described hereafter.

### DETAILED DESCRIPTION

A. Definitions

Unless defined otherwise, all technical and scientific terms used herein have the same meaning as is commonly understood by one of skill in the art to which the subject matter described herein belongs.

As used herein, the terms “heterologous” DNA or gene, or “foreign nucleic acid” are used interchangeably and refer to DNA or RNA that does not occur naturally as part of the genome in which it is present or which is found in a location or locations in the genome that differs from that in which it occurs in nature. Heterologous nucleic acid is generally not endogenous to the cell into which it is introduced, but has been obtained from another cell or prepared synthetically. Generally, although not necessarily, such nucleic acid encodes RNA and proteins that are not normally produced by the cell in which it is expressed. These are referred to herein as heterologous RNAs and heterologous proteins. Any DNA or RNA that one of skill in the art would recognize or consider as heterologous or foreign to the cell in which it is expressed is herein encompassed by the term heterologous DNA. Examples of heterologous DNA include, but are not limited to, DNA that encodes transcriptional and translational regulatory sequences and selectable or traceable marker proteins, such as a protein that confers drug resistance. Heterologous DNA may also encode DNA that mediates or encodes mediators that alter expression of endogenous DNA by affecting transcription, translation, or other regulatable biochemical processes. Heterologous genes or DNA may, for example, refer to a gene or DNA sequence that is not naturally present in yeast. The term “recombinant DNA” may be used to refer to DNA that contains heterologous nucleic acid, including, for example, a gene, or a non-coding region.

As used herein, the term “vector” refers to a polynucleotide construct designed for transduction/transfection of one or more cell types. Selection and use of such vectors are well within the level of skill of the art. Generally, vectors are derived from viruses or plasmids of bacteria and yeasts.

As used herein, “expression” refers to the process by which nucleic acid is transcribed into mRNA and translated into peptides, polypeptides, or proteins. “Expression” may be characterized as follows: A cell is capable of synthesizing many proteins. At any given time, many proteins that the cell is capable of synthesizing are not being synthesized. When a particular polypeptide, coded for by a given gene, is being synthesized by the cell, that gene, or the peptide, polypeptide, or protein it codes for, is said to be expressed. In order to be expressed, the DNA sequence coding for that particular polypeptide must be properly located with respect to the control region of the gene. The function of the control region is to permit the expression of the gene under its control. As used herein, the term “expression vector” includes vectors capable of expressing DNA or RNA fragments that are in operative linkage with regulatory sequences, such as promoter regions, that are capable of effecting expression of such DNA or RNA fragments. Thus, an expression vector refers to a recombinant DNA or RNA construct, such as a plasmid, a phage, recombinant virus or other vector that, upon introduction into an appropriate host cell, results in expression of the cloned DNA or RNA. Appropriate expression vectors are well known to those of skill in the art and include those that are replicable in eukaryotic cells and/or prokaryotic cells and those that remain episomal or may integrate into the host cell genome. As used herein, “expression” includes transcription and/or translation of a polynucleotide sequence. Expressed proteins may also be post-translationally modified.

As used herein, the terms “operative linkage” or “operative association” of heterologous DNA to regulatory and effector sequences of nucleotides, such as promoters, enhancers, transcriptional and translational stop sites, and other signal sequences, refer to the functional relationship between such DNA and such sequences of nucleotides. For example, operative linkage of heterologous DNA to a promoter refers to the physical and functional relationship between the DNA and the promoter such that the transcription of such DNA is initiated from the promoter by an RNA polymerase that specifically recognizes, binds to and correctly transcribes the DNA.

As used herein, the term “promoter region” refers to the portion of DNA of a gene that controls transcription of DNA to which it is operatively linked. A portion of the promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences may be cis acting or may be responsive to trans acting factors. Promoters, depending upon the nature of the regulation, may be constitutive or regulated. The promoters used are those functional in yeast and may include, for example, inducible promoters. The promoters are recognized by an RNA polymerase that is expressed by the host.

As used herein, the term “transcription terminator region” has (a) a subsegment that encodes a polyadenylation signal and polyadenylation site in the transcript, and/or (b) a subsegment that provides a transcription termination signal that terminates transcription by the polymerase that recognizes the selected promoter. The entire transcription terminator may be obtained from a protein-encoding gene, which may be the same or different from the gene, which is the source of the promoter. Transcription terminator regions can be those that are functional in yeast. Transcription terminators are optional components of the expression systems herein, but are employed in preferred embodiments.

As used, the term “nucleotide sequence coding for expression of” or “encoding” a polypeptide refers to a sequence that, upon transcription and subsequent translation of the resultant mRNA, produces the polypeptide.

As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, maintenance of the correct reading frame of a protein-encoding gene to permit proper translation of the mRNA, and stop codons. In addition, sequences of nucleotides encoding a fluorescent indicator polypeptide, such as a green or blue fluorescent protein, can be included in order to select positive clones (i.e., those host cells expressing the desired polypeptide).

As used herein, a heterologous gene causes recombinant yeast to produce a natural product by, for example, encoding a protein that when expressed, is a natural product. Or, the heterologous gene may encode an enzyme that is part of a metabolic process that causes the yeast to produce the desired natural product. More than one heterologous gene may be expressed in the recombinant yeast, each encoding a protein necessary for the metabolic process.

As used herein, the term “concentrated wine” or “concentrated grape juice” or “enhanced wine” is used to refer to wine that has been fermented using recombinant yeast of the present invention, so that the wine has an increased concentration of one or more natural products.

As used herein, the term “grape juice” is used to refer to juice prepared from grapes, for example, grapes cultivated for wine production.

As used herein, the term “wine” is used to describe a product resulting from an alcoholic fermentation of juice or must of grapes or of any other fruit or berries, whether the fermentation occurs spontaneously or it is obtained by the addition of a yeast culture.

Wines that may be produced using the methods of the present invention include red wines, white wines, and rose wines, or sparkling wine versions thereof. Wines of the present invention may be produced using any fruit known to be used to produce wine. In certain embodiments, the wine is made from grapes. Wines of the present invention may be all of one grape varietal, or may include wines of different types of grapes, also called meritage wines. Wine grape varieties represent only a small portion of the more than 600 kinds of grapes. Each grape variety has its own unique combination of characteristics including color, size, skin thickness, acidity, yield per vine, and flavor. Those of ordinary skill in the art may select the appropriate grape to produce the desired type of wine of the present invention. Wines of the present invention may, for example, be derived from fruit, or grapes known to those of ordinary skill in the art. White wines include, but are not limited to, those derived by fermentation of one or more of the following varietals of grapes: Sauvignon Blanc, Chardonnay, Viognier, Pinot Gris, Pinot Blanc, Chenin Blanc, Columbard, Folle Blanche, Grunerveltiner, Malvasia, Marsanne Melon de Bourgogne, Muller-Thurgau, Muscadelle, Palomino, Pedro Ximenez, Gewurztraminer, Riesling, Muscat, Semillon, Traminer, Aligot, Scheurebe, Sylvaner, Ugni Blanc, Verdicchio, and Trebbiano. Red wines include, but are not limited to, those derived by fermentation of one or more of the following varietals of grapes: Pinot Noir, Merlot, Zinfandel, Cabernet Sauvignon, Syrah, Shiraz, Petite Syrah, Sangiovese, Cabernet Franc, Barbera, Barbarossa, Brunello, Cabernet Franc, Carignane, Carmenere, Cinsault, Dolcetto, Durif, Gamay, Gamay Noir, Gamay Beaujolais, Grenache, Grignolino, Malbec, Montepulciano, Mourvedre, Muscat, Nebbiolo, Petite Sirah, Petit Verdot, Pinotage, Pinot Meunier, Tempranillo, tinta Barroca, Tinta Cau, Touriga, Francesa, Touriga Nacional, and Tinta Roriz.

Sparkling and rose wines include, but are not limited to, those derived by fermentation of one or more of the red or white wine varietals listed herein, or known to those of ordinary skill in the art. The listing of varietals is not intended to be exhaustive, and merely provides examples of grapes that may be used in the present invention. Meritage wines may include, for example, two or more different varietals, such as two or more varietals selected from, for example, the preceding lists.

Non-grape fruits include, but are not limited to, plum, elderberry, black currant, plum, peach, blackberry, huckleberry, apricot, banana, blueberry, cherry, cloudberry, goji (wolfberry), gooseberry, pear, raspberry, red currant, rowan, persimmon, pineapple, quince, rose hip, sea-buckthorn, strawberry, watermelon, mangosteen, mango, sweetsop, crowberry, and kiwi. Wines may also be made from fruit flowers, such as dandelion and elderberry flowers. Wines may also be made from rice, potato, rhubarb, and parsnip.

As used herein, the term “transfection” refers to the taking up of DNA or RNA by a host cell. Transformation refers to this process performed in a manner such that the DNA is replicable, either as an extrachromosomal element or as part of the chromosomal DNA of the host. Methods and means for effecting transfection and transformation are well known to those of skill in this art (see, e.g., Wigler et al. (1979) Proc. Natl. Acad. Sci. USA 76:1373-1376; Cohen et al. (1972) Proc. Natl. Acad. Sci. USA 69:2110)). Cells may be transfected in vitro, or may be transfected after administration of the DNA or RNA to a host, for example, by injection.

As used herein, the term “transformation” refers to the transfer of a nucleic acid fragment into the genome of a host cell, resulting in genetically stable inheritance. A “host cell” is a cell that has been transformed, or is capable of transformation, by an exogenous nucleic acid molecule. Host cells containing the transformed nucleic acid fragments are referred to as “transgenic” cells, and organisms comprising transgenic cells are referred to as “transgenic organisms”. For example, transformed yeast may include yeast that is stably transformed with a plasmid that autonomously replicates, or yeast that is transformed with a plasmid or other DNA sequence that is integrated into a yeast chromosome.

As used herein, the term “isolated substantially” pure DNA refers to DNA fragments purified according to standard techniques employed by those skilled in the art (see, e.g., Maniatis et al. (1982) Molecular Cloning: A Laboratory Manual, Cold Spring Harbor Laboratory Press, Cold Spring Harbor, N.Y. and Sambrook et al. (1989) Molecular Cloning: A Laboratory Manual, Cold Spring Harbor Laboratory Press, Cold Spring Harbor, N.Y.).

As used herein, a “culture” means a propagation of cells in a medium conducive to their growth, and all sub-cultures thereof. The term subculture refers to a culture of cells grown from cells of another culture (source culture), or any subculture of the source culture, regardless of the number of subculturings that have been performed between the subculture of interest and the source culture. The term “to culture” refers to the process by which such culture propagates.

As used herein, the term “peptide” and/or “polypeptide” means a polymer in which the monomers are amino acid residues that are joined together through amide bonds, alternatively referred to as a polypeptide. When the amino acids are alpha-amino acids, either the L-optical isomer or the D-optical isomer can be used, the L-isomers being preferred. Additionally, unnatural amino acids such as beta-alanine, phenylglycine, and homoarginine are meant to be included. Standard single and three letter naming conventions for amino acids are used herein.

As used herein, a cloning vector is a DNA molecule that carries foreign DNA into a host cell, replicates inside a bacterial or yeast cell and produces many copies of itself and the foreign DNA. Vectors may contain inducible promoters for the expression of desired peptides, polypeptides, proteins and enzymes. Several types of vectors exist for the purpose of cloning and may in include but are not limited to plasmids, phage, cosmids bacteria artificial chromosomes (BAC), and yeast artificial chromosomes (YAC).

The term “cloning” and “sub-cloning” are used interchangeably and related to the insertion of heterologous DNA into a desired vector for insertion into a cell, bacterial or yeast. General steps of cloning are well known to those skilled in the art and are briefly described below. Vectors and DNA to be cloned are prepared by digestion with restriction enzymes to generate complementary ends facilitating subsequent ligation. One or more fragments of DNA encoding the desired gene or genes may be ligated into the same vector. The heterologous DNA is ligated into the vector with the enzyme DNA ligase. DNA fragments may be ligated into a region of a vector under the control of an inducible promoter. The DNA is inserted into the yeast cells by a process called transformation. Cells containing the heterologous DNA can be screened for by the use of selectable markers such as drug resistance or the expression of fluorescent markers.

As used herein, the term “restriction enzyme digestion” of DNA refers to catalytic cleavage of the DNA with an enzyme that acts only at certain locations in the DNA. Such enzymes are called restriction endonucleases, and the sites for which each is specific is called a restriction site. The various restriction enzymes used herein are commercially available and their reaction conditions, cofactors, and other requirements as established by the enzyme suppliers are used. Restriction enzymes commonly are designated by abbreviations composed of a capital letter followed by other letters representing the microorganism from which each restriction enzyme originally was obtained and then a number designating the particular enzyme. In general, about 1 micrograms of plasmid or DNA fragment is used with about 1-2 units of enzyme in about 20 microliters of buffer solution. Appropriate buffers and substrate amounts for particular restriction enzymes are specified by the manufacturer. Incubation of about 1 hour at 37° C. is ordinarily used, but may vary in accordance with the supplier's instructions. After incubation, protein or polypeptide is removed by extraction with phenol and chloroform, and the digested nucleic acid is recovered from the aqueous fraction by precipitation with ethanol. Digestion with a restriction enzyme may be followed with bacterial alkaline phosphatase hydrolysis of the terminal 5′ phosphates to prevent the two restriction cleaved ends of a DNA fragment from circularizing or forming a closed loop that would impede insertion of another DNA fragment at the restriction site. Unless otherwise stated, digestion of plasmids is not followed by 5′ terminal dephosphorylation. Procedures and reagents for dephosphorylation are conventional as described in Sections 1.56-1.61 of Sambrook, et.al., Molecular Cloning: A Laboratory Manual New York: Cold Spring Harbor Laboratory Press, 1989 (which disclosure is hereby incorporated by reference).

The terms “recovery” or “isolation” of a given fragment of DNA from a restriction digest mean separation of the digest, e.g., on polyacrylamide or agarose gel by electrophoresis, identification of the fragment of interest by comparison of its mobility versus that of marker DNA fragments of known molecular weight, removal of the gel section containing the desired fragment, and separation of the gel from DNA. These procedures are generally well known. For example, see Lawn et al., 1981, Nucleic Acids Res., vol. 9, pp. 6103-6114; and Goeddel et al., 1980, Nucleic Acids Res., vol. 8, p. 4057, which disclosures are hereby incorporated by reference.

As used herein, the term “gene” refers to those DNA sequences which transmit the information for and direct the synthesis of a single protein chain.

As used herein, the term “plasmid” means a vector used to facilitate the transfer of exogenous genetic information, such as the combination of a promoter and a heterologous gene under the regulatory control of that promoter. The plasmid can itself express a heterologous gene inserted therein. “Plasmids” are designated by a lower case p preceded and/or followed by capital letters and/or numbers. The starting plasmids herein are commercially available, are publicly available on an unrestricted basis, or can be constructed from such available plasmids in accord with published procedures. In addition, other equivalent plasmids are known in the art and will be apparent to one of ordinary skill in the art.

The term “ligation” means the process of forming phosphodiester bonds between two nucleic acid fragments. To ligate the DNA fragments together, the ends of the DNA fragments must be compatible with each other. In some cases, the ends will be directly compatible after endonuclease digestion to blunt ends to make them compatible for ligation. To blunt the ends, the DNA is treated in a suitable buffer for at least 15 minutes at 15° C., with about 10 units of the Klenow fragment of DNA polymerase I or T4 DNA polymerase in the presence of the four deoxyribonucleotide triphosphates. The DNA is then purified by phenolchloroform extraction and ethanol precipitation. The DNA fragments that are to be ligated together are put in solution in about equimolar amounts. The solution will also contain ATP, ligase buffer, and a ligase such as T4 DNA ligase at about 10 units per 0.5 g of DNA. If the DNA is to be ligated into a vector, the vector is first linearized by digestion with the appropriate restriction endonuclease(s). The linearized fragment is then treated with bacterial alkaline phosphatase, or calf intestinal phosphatase to prevent self-ligation during the ligation step.

As used herein, the term “preparation of DNA from cells” means isolating the plasmid DNA from a culture of the host cells. Commonly used methods for DNA preparation are the large and small scale plasmid preparations described in sections 1.25-1.33 of Sambrook et al., supra, which disclosure is hereby incorporated by reference. After preparation of the DNA, it can be purified by methods well known in the art such as that described in section 1.40 of Sambrook et al., supra, which disclosure is hereby incorporated by reference.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, genomic RNA, mRNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P—NH₂) or a mixed phosphoramidate-phosphodiester oligomer. (Peyrottes et al. (1996) Nucleic Acids Res. 24: 1841-8; Chaturvedi et al. (1996) NucleicAcids Res. 24: 2318-23; Schultz et al. (1996) Nucleic Acids Res. 24: 2966-73). A phosphorothioate linkage can be used in place of a phosphodiester linkage. (Braun et al. (1988) J. Immunol. 141: 2084-9; Latimer et al. (1995) Molec. Immunol. 32: 1057-1064). In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. Reference to a polynucleotide sequence (such as referring to a SEQ ID NOs. 1-6) also includes the complement sequence.

The following are non-limiting examples of polynucleotides: a gene or gene fragment, exons, introns, genomic RNA, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

As used herein, the term “under transcriptional control” refers to a term well understood in the art and indicates that transcription of a polynucleotide sequence depends on its being operably (operatively) linked to an element that contributes to the initiation of, or promotes, transcription. “Operably linked” refers to a juxtaposition wherein the elements are in an arrangement allowing them to function.

“Replication” and “propagation” are used interchangeably and refer to the ability of an VSV vector of the invention to reproduce or proliferate. These terms are well understood in the art. For purposes of this invention, replication involves production of VSV proteins and is generally directed to reproduction of VSV. Replication can be measured using assays standard in the art. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

The practice of the present invention will employ, unless otherwise indicated, conventional techniques of molecular biology (including recombinant techniques), microbiology, cell biology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

### Natural Products

Natural products are any product that may be found in a living organism, such as, for example, an animal or a plant. For example, in certain embodiments, natural products are any product expressed in a living organism. For example, natural products may be any product from a plant. Although natural grape plant products are used in many examples in the present application, those of ordinary skill in the art can readily apply these examples to natural products present in other plants. Various natural products may be used to enhance the flavor of wine produced by the methods of the present invention. These natural products may already be present in conventionally-produced wine, wine produced using conventional yeast or “non-concentrated wine,” and the concentrations of the natural products is increased using the present methods. Thus, the terms “conventionally-produced wine” or “non-concentrated wine” refer to wine that has not been produced using recombinant yeast that secrete natural products. Also, for example, natural products that are not present in the conventionally produced wine, or present at levels difficult to detect, may be added to enhance the flavor of the wine. Examples of natural products are listed in Table 1, and are also discussed below:

Esters—Acetyl-transferases may be modified to improve efficiency to make either acetyl-alcohols or—higher alcohols . . . or to acetyl-CoA. Carboxyl methyl transferases may also be used to create methyl esters and for alcohol, higher alcohols and acids (wintergreen flavor).

Isoprenoids—Various isoprenoids may be secreted by recombinant yeast, including, for example, terpenes, such as, for example, sesquaterpenes, norisoprenoids, and, related alcohol versions of these compounds. A cartenoid creation pathway may be used to produce these compounds.

Phenols—The simplest forms of phenols to produce in recombinant yeast may be, for example, resveratrol and metabolites thereof (pysotanol). Other phenols that may be produced using the methods of the present invention include, for example, the flavons, which have both health and taste characteristics. Other examples of phenols that may be produced using the methods of the present invention include the anthrocyanins which can involve many genes (7+) and then polymers of these compounds. Hybrid phenols with isoprenoids may also be created.

Other examples of natural products that may be used in the present invention include acids, such as, for example, tartaric acid and malic acid. Also, higher alcohols and pyrazines may be used if they impart enhanced flavor characteristics to wine.

Natural products may be present in plants other than grapes. For example, natural products present in oak may be used to enhance the oak barrel characteristics of wine. The methods of the present invention may also be used to impart an oak barrel flavor to wine that has not been actually aged in an oak barrel. Other characteristics often found that add to the complexity of wine flavor can be imparted to wine. For example, natural products may be used to add a raspberry, coffee, chocolate, or cherry aspect to the wine flavor. Further examples of natural products that may be used in the present invention are discussed in the Examples.

Natural products include, for example, those listed in Table 2.

TABLE 2

| Type of Natural Product | Examples |
|---|---|
| Acids | Tartaric |
| | Malic |
| | Lactic |
| | Succinic |
| | Citric |
| | Fumaric |
| | Ascorbic |
| | Acetic |
| Terpenes | Sub-forms: simple-hydrocarbon, aldehydes, |
| | alcohols, polyols, glycocides |
| | Monoterpenes |
| | Sesquiterpenes |
| | Diterpenes |
| | Are most commonly associated with aroma in |
| | Muscat wines: linalool, geraniol, nerol, |
| | limonene, citronellol, and alpha-terpineol. |
| Norisoprenoids | Sub-forms: simple, glycoside, aromatic, non- |
| | aromatic |
| | Damascenone |
| | Vitispiranes |
| | Megastigmatrieones |
| Oak-related | Lactones - for example, methyl-octalactones: |
| Natural Products | alpha-methyl-beta-octalactone, â-methyl-ã- |
| | octalactone |
| | eugenol |
| | vanillin, vanillyl alcohol |
| | cyclotene |
| | ethoxylactone |
| | maltol, hydroxymaltol, dihydromaltol, |
| | syringaldehyde |
| | sinapaldehyde |
| | furfurals |
| | lignin |
| | hemi-cellulose |
| | tannins: Gallitannins, ellagitannins, |
| Phenols | Both flavonoids and non-flavonoids |
| | Sub classes non-flavonoids: |
| | Hydroxycinnamates, benzoic acids, |
| | stilbenes |
| | Sub classes of flavonoids: flavonols, |
| | flavan-3-ols, proanthocyanidins |
| | (condensed tannins), and anthocyanins |
| | Examples include, but are not limted |
| | to: flavonols: quercetin-glycosides, |
| | flavonol-glycosides (most wine |
| | flavonols occur as glycosides however |
| | galactosides, and glucuronides with |
| | some rare cases of diglycosides are |
| | found) |
| | Examples include, but are not limited |
| | to: flavan-3-ols: catechin, epicatechin, |
| | epigallocatechin, and epicatechin |
| | gallate (agallic acid ester) |
| | Proanthocyanidins, or condensed tannins, are |
| | oligomers and polymers of flavan-3-ol |
| | monomers |
| | Anthocyanins |
| | Examples include, but are not limited |
| | to anthocyanins: Delphinidin, |
| | Cyanidin, Petunidin, Peonidin, and |
| | Malvidin |
| | Hydroxycinnamates: caffeic, coumaric, and |
| | ferulic acid (as esters: caftaric, coutaric, and |
| | fertaric acid) |
| | Benzoic acids: gallic or ellagic acid |
| | Stilbenes: Resveratrol |
| | Tannins: procyanidins, prodelphinidins. |
| | Other polymerizations of the above with all |
| | sets of connectivity |
| Alcohols | Glycerol |
| | Polyols (alditols (erythritol, xylitol, arabitol, |
| | mannitol, sorbitol, and myo-inositol) |
| | 2,3 butanediol |
| | fusel oils |
| Pyrazines | Methoxypyrazines: |
| | Isobutylmethoxypyrazine |
| | sec-butylmethoxypyrazine |
| | isopropylmethoxypyrazine |
| Esters | Ethanol, acetyl, etc . . . via acetyl-CoA |
| | Ethyly acetate |

Expression of Plant Genes in Microbes

Various methods for the expression of various plant genes in microbes, such as bacteria and yeast, are known to those of ordinary skill in the art. Examples of bacterial expression of heterologous genes coding for enzymes related to natural product metabolic pathways are provided in Louie, G. V., et al., (2007) Structure and Reaction Mechanism of Basil Eugenol Synthase, PLoS ONE 2(10): e993.doi:10.1371/journal.pone.0000993; Koeduka, T., et al., Proc. Natl. Acad. Sci. 103:10128-33 (2006) (eugenol and isoeugenol); and Starks, C. M., et al., Science 277:1815 (1997) (terpene). Methods for identifying enzymes involved in the synthetic pathways for natural products are discussed in, for example, B. J. Nikolau and E. Syrkin Wurtele (eds.) Concepts in Plant Metabolomics 169-182 (Springer, 2007). The expression of plant genes in yeast is exemplified in Allen, S. C., et al., FEBS J., 274:5586-99 (2007) (ricin A); Han, Y., et al., Appl. Environ. Microbiol. 65:1915-8 (1999) (phytase); and Hamilton, A. J., et al., Proc. Natl. Acad. Sci. 88:7434-7437 (1991). In addition, a recombinant wine yeast strain expressing the gene coding for beta-(1,4)-endoxylanase from Asperigillus nidulans under the control of the yeast actin promoter secreted active xylanase enzyme into the culture medium (Ganga, M. A., et al., Int. J. Food Microbiol.47:171-8 (1999). To optimize expression in yeast, preferred codons may be used in the gene coding for the natural product metabolic enzyme. The use of yeast gene codon optimization is known to those of ordinary skill in the art and is discussed in, for example, Kotula, L., and Curtis, P. J., Bio/Technology 9:1386-89 (1991).

Kits

The present invention further provides kits comprising the recombinant yeast of the present invention, in a suitable container. For example, such kits may comprise one or more recombinant yeast strains, each transformed with a DNA sequence comprising a heterologous gene that causes the yeast to secrete a natural product. More than one yeast strain may be included, each increasing the relative concentration of a different natural product when used to ferment wine.

The kit may further comprise one or more small vessels used to produce small batches of natural product enhanced wine.

The kits of the present invention may also comprise instructions for performing one or more methods described herein and/or a description of one or more compositions or reagents described herein for producing natural product enhanced wine. Instructions and/or descriptions may be in printed form and may be included in a kit insert. A kit may, for example, include a written description of an Internet location that provides such instructions or descriptions.

These instructions may, for example, provide instructions for fermenting grape juice with the recombinant yeast. These instructions may, for example, provide instructions for mixing and testing various fermented grape juice mixtures. The instructions may, for example, provide suggested mixture ratios to test, or suggestions as to amounts of natural product concentrated wine to add to conventionally produced wine.

The kits of the present invention may also comprise one or more of the components in any number of separate containers, packets, tubes, vials, microtiter plates and the like, or the components may be combined in various combinations in such containers.

Kits may, instead of including recombinant yeast, comprise DNA plasmids or other vectors of the present invention that may be used to transform yeast to obtain the recombinant yeast of the present invention.

### EXAMPLES

The following examples are provided to illustrate certain embodiments of the invention and are not limiting.

### Example 1—Recombinant Yeast

Examples as to how to transform yeast with a desired gene or genes are well known to those skilled in the art and an example is briefly described below.

Competent yeast cells are prepared prior to transformation by inoculating starter yeast cultures (˜50 ml) containing desired growth media and grown overnight. Cells may be supplemented with yeast extracts following overnight growths. Cells are harvested by centrifugation, washed, and resuspended in appropriate buffer (generally pH neutral) containing 100 mM Lithium Acetate, beta-mercaptoethanol and carrier DNA. Competent cells are aliquoted into appropriate containers with desired DNA for transformation and incubated at 30° C. for 30 minutes. Desired DNA can contain one or more desired genes or one or more plasmids containing desired genes as necessary for a particular expression product or as part of a particular pathway to achieve synthesis of desired product. The plasmid may also comprise a promoter or other DNA sequence operably linked to the DNA sequence comprising the heterologous gene such that the heterologous gene is overexpressed in the recombinant yeast. Polyethylene Glycol is added and the mixture is further incubated at 30° C. for up to 30 minutes. The Yeast/DNA mixture is then heat-shocked at 45° C. for 15 minutes and subsequently centrifuged to pellet cells. The PEG layer is removed and transformed cells may be plated on solid or liquid medium as necessary, containing appropriate selection markers.

Yeasts may be any form including but not limited to: active (ADY) or inactive dry yeasts (IDY), liquid yeasts and pitchable yeasts. Yeast may be isolated purified yeasts or of a mixed population strain. Common wine making yeasts include but are not limited to: Saccharomyces Banyus, Saccharomyces Cerevisiae and Saccharomyces Fermentati. Common yeast strains for winemaking include, but are not limited to, the following examples:

43, 71B-1122, AC-, AMH (Assmanshausen), BA11, BDX, BGY (Burgundy), BM45, BRL97, CSM, Beaujolais, Bordeaux, Bourgovin, CY3079, Cabernet, Chablis, Champagne, Chardonnay, Chianti, Cold Fermentation, Côte des Blancs, DV10, D47, EC-1118, Epernay, F33, Flor Sherry, ICV-D21 (Languedoc), ICV-D254, ICV-D80, ICV-D21, ICV-D47 (Côtes-du-Rhône), ICV-D80 (Côte Rôtie), ICV-D254, ICV-GRE, K1-V1116 (Montpellier), L2056, L2226, Liebfraumilch, MO5, M1, M2, Madeira, Montrachet, Muscat, Pasture Red, Pasteur White, Premier Curvee, Pinot Noir, Port, Prise de Mousse, QA23, R2, RA17, RC-212 (Bourgovin), R-HST, Rudisheimer, S6U, Sake #9, Simi-White, Syrah, T73, T306, W15, W27, W46, Eau de Vie—(Water of Life), Sweet Mead/Wine,730,, Merlot, , English Cider, Hock, Riesling, Rosé, SB 23 Super Yeast, Sauternes, Sauvignon, Sherry, Steinberg and Tokay.

More than one type of yeast may be used in the same batch to produce the wines and concentrated wines of the present invention.

### Example 2: Example with a Natural Product

It is known in the art that yeasts can be designed to produce any one of a number of natural products. This is accomplished by incorporation into yeast a gene or genes responsible for the production of a natural product or a peptide, polypeptide, protein or proteins involved in a metabolic pathway responsible for the production of a natural product. Engineered yeasts are constructed by incorporating a gene or genes of interest by sub-cloning desired gene, or genes in an inducible region of the vector being used. Several examples of engineered yeasts capable of producing natural products currently exist. Examples of natural product, producing systems include but are not limited to the following examples.

For example genes involved in the methylerythritol phosphate (MEP) pathway to isoprene were recently identified (Kayser O., et al. , Appl. Microbiol. Biotechnol. 2007, July 75(6):1377-84 (e-pub Apr. 26, 2007)). Expression of these genes identifies the function of several enzymes in the MEP pathway and demonstrate a marked increase in the production of isoprene. Another example is demonstrated in Chappell et al., Plant Physiol. 2008, May 8 (e-pub ahead of print) and Takahashi, S., et al., Biotechnology and Bioengineering 97:170-181 (2007). Genes responsible for the over production of a key terpene intermediate farnesyl diphosphate (FFP) are engineered into yeast and co-expressed with terpene synthetase genes (converting FFP to sesquiterene) and terpene hydroxylase. The expression of these genes in the engineered yeast demonstrates marked production of a hydroxylated terpene. These examples demonstrate the ability to produce terpenoid precursors, isopentenyl diphosphate, dimethlyallyl diphosphate for producing single entity, complex and stereochemically unique terpenes and terpenoids.

### Example 3: Fermentation

Grape juice prepared from grapes grown for wine cultivation may be prepared using standard methods known in the art. The majority of the processed grape juice (pre-fermentation) is fermented via conventional methods using traditional non-transformed yeast, while the remainder of grape juice is transferred to a number of smaller vessels for the fermentation with any of the recombinant yeast of the present invention. Typically, one strain of transformed yeast is used per smaller vessel. In other examples, more than one recombinant strain may be used in the same smaller vessel. In other examples, a recombinant strain may be in the same smaller vessel as a non-recombinant strain, if, for example, the non-recombinant strain is also found to assist in proper fermentation of the grape juice, or if it is found to improve the final product, for example, if it enhances the flavor. Many strains of transformed yeast can be used to produce a wide range of flavor enhanced concentrated wine. Fermentation in the smaller vessels may utilize technology or equipment that allow for high density cell growth and/or increased gene expression and hence natural product production.

The general techniques for production of wine are known to those of ordinary skill in the art, and are also available in general references, such as, for example Margalit, Yair, Concepts in Wine Chemistry (The Wine Appreciation Guild, South San Francisco) 2004 (ISBN 1-891267-74-4); and Margalit, Yair, Concepts in Wine Technology (The Wine Appreciation Guild, South San Francisco) 2004 (ISBN 1-891267-51-5).

### Example 4: Production of Enhanced Flavor Wine

Processing:

Following fermentation, the recombinant and non-recombinant yeast, as applicable, may be filtered out of the fermented grape juice concentrate.

Mixing:

The wine fermented with the transformed yeast, the concentrated wine, is mixed with that fermented with the conventional yeast in proportions that create preferred tastes for particular wine types. More than one concentrated wine may be used in the mixture. Or, concentrated wines may be mixed without conventionally produced wine. This process could involve random or organized mixing and tasting in varied and scaled proportions. Each wine flavor created by the transformed yeast offers the option to increase or add (for the first time) the relative concentration of such natural product in the wine fermented with the conventional yeast.

The entirety of each patent, patent application, publication, document and sequence (e.g., nucleotide sequence, amino acid sequence) referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

What is claimed is:

1. A method of producing wine, comprising mixing at least two fermented grape juices, wherein at least one of said fermented grape juices is fermented by recombinant yeast transformed with a DNA sequence comprising a heterologous gene, wherein the expression of said heterologous gene causes the yeast to secrete a flavor-enhancing natural product.

2. The method of claim 1, wherein said gene encodes a natural product.

3. The method of claim 1, wherein said gene encodes a protein that produces a natural product.

4. The method of claim 1, wherein said recombinant yeast is transformed with DNA coding for at least two heterologous proteins, wherein the expression of said heterologous proteins causes the yeast to secrete at least two different natural products.

5. The method of claim 1, wherein said natural product is a product that is produced by a grape plant.

6. The method of claim 1, wherein said natural product is a product that is present in grapes.

7. The method of claim 1, wherein said natural product is a product that is present in wood.

8. The method of claim 8, wherein said wood is oak wood.

9. The method of claim 8, wherein said natural product is selected from the group consisting of lactones, eugenol, vanillin, vanillyl alcohol, cyclotene, ethyoxylactone, maltol, hydroxymaltol, dihydromaltol, syringaldehyde, sinapaldehyde, furfural, lignin, hemi-cellulose, and tannin.

10. The method of claim 1, wherein said natural product is selected from Table 2.

11. The method of claim 1, wherein at least 2 fermented grape juices are mixed, and two of said grape juices are fermented by recombinant yeast transformed with a DNA sequence comprising a heterologous gene, wherein the expression of said gene causes each recombinant yeast to secrete a different flavor-enhancing natural product.

12. The method of claim 1, wherein the grape juice fermented by recombinant yeast is fermented in a separate vessel.

13. A wine produced by the method of claim 1.

14. A method of enhancing the flavor of wine, comprising fermenting grape juice using a recombinant yeast strain that secretes a natural product to obtain an enhanced wine concentrate.

15. The method of claim 14, further comprising adding said enhanced wine concentrate to wine to produce an enhanced-flavor wine.

16. A wine produced by the method of claim 15.

17. The method of claim 15, wherein more than one enhanced wine concentrate is added to said wine, wherein the recombinant yeast strains used to produce each of said enhanced wine concentrates are different.

18. The method of claim 15, further comprising
a) titrating different amounts of at least one enhanced wine concentrate into individual portions of wine; and
b) testing each portion of wine to determine the ratio of enhanced wine concentrates and wine that has an enhanced flavor.

19. The method of claim 18, wherein said testing comprises tasting.

20. The method of claim 18, further comprising preparing a larger batch of enhanced flavor wine using the ratios determined from the method of claim 18.

21. A recombinant yeast comprising a DNA sequence comprising a heterologous gene, wherein the expression of said gene causes said yeast to secrete a natural product.

22. A method of increasing the concentration of a natural product in wine, comprising
a) fermenting grape juice with a recombinant yeast transformed with a DNA sequence comprising a heterologous gene, wherein the expression of said heterologous gene causes the yeast to secrete said natural product; and
b) adding the fermented grape juice to wine, thereby increasing the concentration of said natural product in said wine.
